(19) United States
(12) Patent Application Publication
(10) Pub. No.: US 2024/0325379 A1
(43) Pub. Date: Oct. 3, 2024

(54) LOW-DOSE NALTREXOL AND USES THEREOF

(71) Applicant: AETHER THERAPEUTICS INC., Austin, TX (US)

(72) Inventor: Wolfgang SADEE, Ross, CA (US)

(21) Appl. No.: 18/576,161

(22) PCT Filed: Jun. 24, 2022
(86) PCT No.: PCT/US2022/034828

§ 371 Date: Jan. 3, 2024

Related U.S. Application Data
(60) Provisional application No. 63/218,619, filed on Jul. 6, 2021.

(51) Int. Cl. A61K 31/485

(52) U.S. Cl. CPC A61K 31/485

(57) ABSTRACT

Provided herein is a 6β-naltrexol formulation at dosages sufficient for the treatment of a condition involving upregulated basal signaling of the μ-opioid receptor (MOR) system.

### FIELD OF THE INVENTION

The invention is directed to modulators of opioid addiction to enhance, for example, opioid pain therapies and treat behaviors and traits involving upregulated μ-opioid receptor signaling.

All publications, patents, patent applications, and other references cited in this application are incorporated herein by reference in their entirety for all purposes and to the same extent as if each individual publication, patent, patent application or other reference was specifically and individually indicated to be incorporated by reference in its entirety for all purposes. Citation of a reference herein shall not be construed as an admission that such is prior art to the present invention.

### BACKGROUND OF THE INVENTION

Opioid addiction is a major societal burden. The treatment of pain with opioid drugs is highly effective; however, clinical utility is sharply limited by the development of opioid addiction, which has resulted in a broad crisis, with mounting overdose deaths as a main cause of mortality in the US. In addition, peripheral and central adverse effects include hyperalgesia, opioid induced bowel dysfunction, immune dysfunction, osteopenia, and more. Numerous avenues are being pursued to uncouple opioid analgesia from its deleterious effects, but none has proven highly effective. This invention presents a novel approach selectively to suppress main hallmarks of addition, such as dependence, without diminishing the analgesic opioid effects. When implemented clinically, this approach has outstanding potential to address the opioid crisis, improve pain therapy with opioid drugs, and treat conditions involving dysregulated opioid signaling.

Compulsive addictive behaviors include drug addiction, eating disorders, excessive gambling, and more. Drug addiction involves alcohol, opioids, nicotine, cocaine, other stimulants, and more (42). Taken together, these disorders represent a major societal burden. The opioid signaling system—and specifically the p opioid receptor (MOR)—appears to be involved in many of these disorders, either directly or indirectly (37), leading to therapies aimed at alleviating such behaviors with opioid antagonists such as naltrexone to block opioid substances directly, or to block the contribution of endogenous opioids to addictive behaviors (40). A prominent example of the latter is the treatment of alcohol binge drinking with naltrexone (38,39). MOR also plays a key role in regulating neuropathic pain pathways, intestinal activity, bone remodeling, renal functions and more.

The μ opioid receptor (MOR) plays a key role in multiple physiological and pathophysiological conditions and behaviors (37,42). These include addictive behaviors (opioids, alcohol, nicotine, stimulants, and other drug use disorders) (4,37), opioid induced hyperalgesia (18,19), opioid induced bowel dysfunction, chronic neuropathic pain, osteopenia, immune functions, cognitive functions and memory loss, eating disorders, compulsive gambling, and more. Upregulated persistent basal MOR signaling (receptor signaling even in the absence of an endogenous ligand or an opioid drug) has been shown to occur with continued activation by agonists, thereby, shifting the inactive MOR-μ to its spontaneously active state MOR-μ*. While initially serving to suppress hyperalgesia and neuropathic pain caused by inflammation (4,5), prolonged MOR-μ* activity may contribute to hyperalgesia and tolerance (15,18,19) and has been invoked as a hallmark of opioid dysregulation and a key to opioid dependence (1,2,6,7,17). A novel treatment strategy is offered with use of compounds that specifically and potently reverse enhanced basal MOR-μ* signaling driving the opioid dependent state, at doses below those that block opioid analgesia or cause withdrawal—thereby selectively modulating opioid addiction—a mechanism discussed in detail by Sadee et al. (16). The same processes appear to be involved in opioid tolerance and hyperalgesia, and possibly drug seeking/craving behavior, all aspect critical to driving opioid addiction, thereby, reducing clinical utility of opioid pain therapy. The term ‘addiction modulator’ is used here for compounds with such selective effects.

A need exists in the art, therefore, for an effective strategy to treat, for example, opioid addiction to enhance opioid pain therapies and treat behaviors and traits involving upregulated μ-opioid receptor signaling with low dosages of an addiction modulator.

### SUMMARY OF THE INVENTION

The novel model of the MOR receptor (16, and references therein)—accounting for otherwise inexplicable observations—supports the use of 6β-naltrexol and chemical compounds with similar characteristics for treating generally addictive behaviors involving μ opioid receptor signaling and dysregulation, given in low doses gradually to stabilize a receptor site equilibrium prevalent in the non-dependent state, without acting as a typical opioid antagonist. One can consider the effects of continuous elevated stimulation of opioid receptors (dysregulation) by endogenous opioids in other drug use disorders or compulsive behaviors to have similar effects compared to those caused by ingested opioid drugs. Naltrexone is an effective opioid antagonist to reduce alcohol binge drinking but it causes aversion in a substantial portion of subjects leading to low compliance—likely attributable to an upregulated basally active MOR-μ* state at which naltrexone acts as an inverse agonist (it blocks signaling by MOR-μ*, in contrast to 6β-naltrexol, which does not). On the other hand, and in contrast to naltrexone, 6β-naltrexol is proposed to facilitate conversion of MOR-μ* back to MOR-μ, an effect that can occur at low receptor occupancy (no effect on analgesia) if the ligand is present over long time periods. Maintaining a MOR-μ-MOR-μ* equilibrium characteristic of the opioid naïve non-dependent state would overcome the disadvantages of naltrexone and enable novel treatment options for numerous conditions associated with addictive behaviors and traits involving MOR signaling, at low 6β-naltrexone doses (LD-6BN) that do not interfere with intended physiological or pharmacological opioid functions. LD-6BN could optimally serve to prevent or reverse dependence and hyperalgesia where potent prolonged pain medication is needed (e.g., cancer, sickle cell anemia, etc), resulting in enhanced pain treatment with lower side effects. LD-6BN is also anticipated to alleviate chronic neuropathic pain and inflammatory pain that is typically refractory to opioid analgesics, and to alleviate behaviors indirectly promoted by dysregulated MOR signaling (e.g., compulsive addictive behaviors). The property of LD-6BN to facilitate conversion of MOR-μ* to MOR-μ represents a novel mechanism for drug ligands, and therefore, 6BN must be considered one example of a new class of compounds that must meet the following requirements: have neutral antagonist properties, facilitate conversion of MOR-μ* to MOR-μ, bind potently to MOR, and be retained at the receptor for extended time periods. 6β-naltrexamide appears to meet these criteria.

### BRIEF DESCRIPTION OF THE FIGURES

FIG. 1 provides data showing prevention of withdrawal behavior in methadone-treated animals.

FIG. 2 shows a model of MOR conformations.

FIG. 3 shows blood and brain levels of 6BN after s.c. doses of 6BN.

### DETAILED DESCRIPTION OF THE INVENTION

It is to be understood that the descriptions of the present invention have been simplified to illustrate elements that are relevant for a clear understanding of the present invention, while eliminating, for the purpose of clarity, many other elements found in typical pharmaceutical compositions and methods of stabilization. Those of ordinary skill in the art will recognize that other elements and/or steps are desirable and/or required in implementing the present invention.

However, because such elements and steps are well known in the art, and because they do not facilitate a better understanding of the present invention, a discussion of such elements and steps is not provided herein. The disclosure herein is directed to all such variations and modifications to such elements and methods known to those skilled in the art. Furthermore, the embodiments identified and illustrated herein are for exemplary purposes only, and are not meant to be exclusive or limited in their description of the present invention.

Many studies had characterized 6β-naltrexol (also referred to herein as 6BN) as a neutral antagonist at MOR, but with only moderate potency, and moderate peripheral selectivity (1-3,7,13) with clinical uses such as treating opioid indued bowel dysfunction and reduction of abuse liability because of 6BN's long half-life and accumulation to high levels under abuse conditions (47). The structure of 6β-naltrexol is shown below:

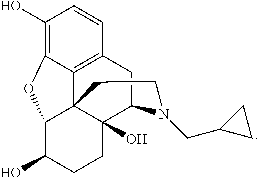 [SMILES: Oc1ccc2c3c1O[C@H]1[C@H](O)CC[C@@]4(O)[C@@H](C2)N(CC2CC2)CC[C@]314]

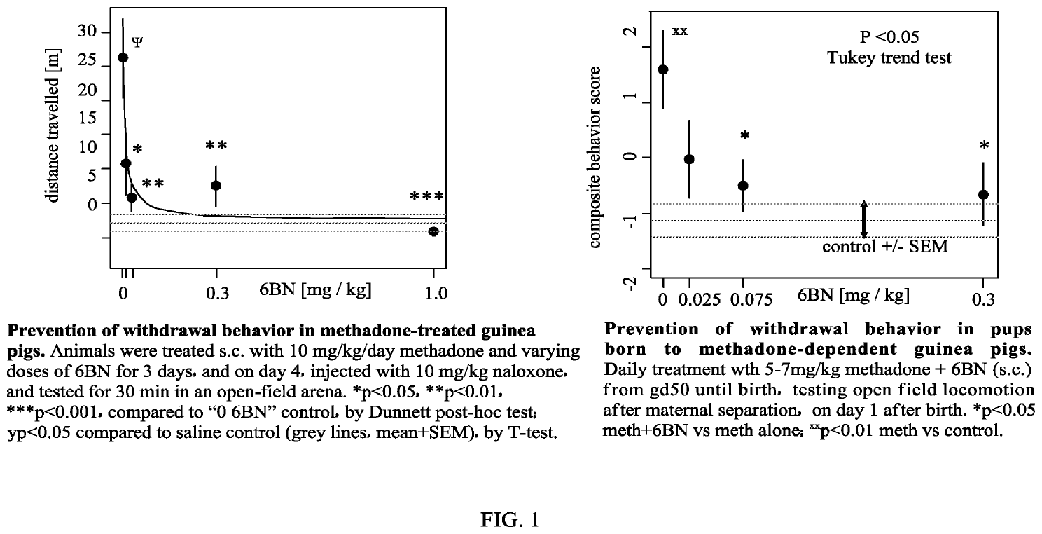

However, to act as a MOR antagonist (blocking analgesia or causing withdrawal), 6BN requires doses higher by more than an order of magnitude than those now shown by us to prevent opioid dependence (30). It is demonstrated in animal models that low-dose 6β-naltrexol reverses the dependent state with high potency, at doses that do not interfere with opioid analgesia, nor cause withdrawal by blocking MOR signaling (30), illustrated in FIG. 1. The inventor has proposed a novel mechanism by which MOR ligands such as 6β-naltrexol, and its analogues including 6β-naltrexamide, can exert entirely different effects at different dose levels at the same receptor (30). This novel mechanism of action leads to a novel approach in clinical applications of low-dose 6β-naltrexol (LD-6BN), treating numerous pathophysiological and behavioral conditions involving upregulated MOR-μ* activity, without the need to block opioid signaling by endogenous opioids physiologically or by opioid drugs, which would require higher 6BN doses when 6BN is intended to serve as a neutral antagonist. As the proposed mechanism of LD-6BN is non-competitive versus opioid agonists (binding to a different receptor conformation), dosing of LD-6BN is independent of agonist potency, efficacy, or dose. The high potency of 6BN for preventing opioid dependence arises from the non-competitive interaction with MOR-μ* and the low fractional receptor occupancy sufficient to achieve this effect. Therefore, this invention differs conceptually and practically from clinical uses described for 6BN when considered a neutral opioid antagonist, which requires titrating doses up to higher levels (47).

It has been shown that naltrexol, naloxol and naltrexamine, and their C-6-O or C-6-N derivatives are neutral antagonists expected to cause less severe withdrawal in opioid dependent subjects with low potency (this effect requires antagonism of opioid agonists) (1-3,6,7). While 6β-naltrexol has shown promise for treating opioid use disorder (20,21), showing advantages over naltrexone (6-8), these 6BN analogs display similar molecular characteristics (47) and are included as drug candidates in the present invention.

Thus, non-limiting embodiments of the present invention include:
1. A method for the treatment with an addiction modulator of conditions involving upregulated basal signaling of the μ opioid receptor (MOR) system in an individual in need thereof comprising administration to the individual of a therapeutically effective amount of 6β-naltrexol. Such conditions include but are not limited to drug use disorders (opioid, alcohol, nicotine cannabinols, stimulants), opioid-induced hyperalgesia, chronic neuropathic pain, and compulsive behaviors including anorexia, binge eating, gambling, excessive sexual behaviors, and more.
2. The method as above, wherein the 6β-naltrexol and naloxol derivatives and analogs include those specified in U.S. Pat. No. 6,713,488 B2, U.S. Pat. Nos. 8,748,448, 8,883,817B2, 9,061,024B2, and EP2214672.
3. The method as above, wherein the opioid antagonist is administered in low doses insufficient to block conventional agonist effects in the central nervous system (analgesia, respiratory depression), but sufficient gradually to bind and deplete the MOR-μ* site which is proposed to drive or sustain drug dependence (1-3, 17,18), chronic neuropathic pain, hyperalgesia, and compulsive behaviors.
4. The method as above, wherein the dose of 6β-naltrexol in humans ranges from 0.001 to 3.0 mg, given orally, intravenously, intranasally, transdermally, subcutaneously, or similar routes, in solution, solid drug formulations, sustained release formulations, and more. Trans-species scaling in dosing will allow application in animals.
5. A method to identify long-acting, neutral μ opioid antagonists binding with high affinity to MOR-μ* without blocking MOR-μ* signaling, acting at low doses selectively as modulators of dysregulated opioid signaling, gradually reducing the level MOR-μ* by accelerating reversal of MOR-μ* to the resting or reserve MOR states. Such assays can be performed by MOR binding studies in vitro and in vivo, by labeling high affinity sites with labeled 6β-naltrexol (e.g., ³H or D, or ¹¹C for in vivo PET studies), and by pharmacological assays outlined in the specifications of the patents and cited references expressly incorporated herein by reference. These criteria serve to identify novel compounds with the therapeutic attributes described above.

The novel finding that low-dose 6β-naltrexol (LD-6BN, <3 mg estimated total oral dose in humans) potently and selectively modulates key aspects of opioid addiction, by reversing long-lasting upregulation of MOR-μ* signaling (30), leads to clinical applications distinct from those described previously for 6β-naltrexol's function as a neutral antagonist, designed to block MOR signaling at higher doses (1-3, 47). These include pain therapy with use of opioid analgesics, when combined with low-dose 6β-naltrexol, having reduced addiction liability and enhanced efficacy, better tolerated opioid maintenance therapies, facilitated opioid weaning/detoxification, and prevention of neonatal opioid withdrawal syndrome, all at doses below doses that block opioid analgesia or cause withdrawal, or interfere with ongoing opioid maintenance therapies (e.g., methadone and buprenorphine). Opioid treatment of severe pain often requires high doses over extended time periods, for example in cancer management (33,34) and sickle cell anemia (35,36); yet, such pain management strategies remain suboptimal. High dose opioid analgesics in cancer lead to multiple adverse effects, including constipation and potentially immune dysregulation, and cognitive decline, whereas in sickle cell anemia patients, high doses cause tolerance and dependence, and pruritis—conditions under which LD-6BN could improve pain management with reduced adverse effects.

Several pain conditions are projected to be sensitive to LD-6BN. Neuropathic pain, allodynia, and hyperalgesia in transition to chronic pain appear to involve elevated MOR-μ* signaling in peripheral sensory neurons and nociceptors (4,15,18,19). It is therefore proposed that LD-6BN can facilitate reversal of these pain conditions, often triggered by inflammatory stimuli and poorly responsive to opioids, and treatable with low-dose naltrexone (26,27), a treatment that can be markedly improved by substituting naltrexone with LD-6BN.

In addition, dysregulation of opioid signaling via endogenous opioid peptides has been invoked in diverse drug use disorders, most notably alcohol use disorder (37-40), and obsessive-compulsive and impulse-control behaviors (42), all of which are included as clinical targets for such opioid addiction modulators. Drug use disorder with opioid signaling involvement include alcohol, nicotine, and cannabinoids, with naltrexone considered a possible pan-addiction treatment (40), but with limited efficacy, likely owing to its potent inverse agonist activity.

Taken together, the unique properties of LD-6BN and its congeners as addiction modulators are novel and predict novel clinical applications without need to titrate 6BN dosages up to levels at which 6BN acts as a neutral antagonist (e.g., for treating opioid induced constipation; >3 mg total oral dose in humans). Long-term opioid use or continuous stimulation via endogenous opioids leads to dependence, risk of addiction, and deleterious adverse effects, such as hyperalgesia and opioid induced bowel dysfunction, at least in part a result of upregulated sustained MOR-μ* activity. Such events can be reversed, prevented, or alleviated with LD-6BN, in a dosage range deemed clinically safe. Indeed, low-dose naltrexone had been proposed as a treatment of various inflammatory pain conditions and to improve pain therapy with opioid analgesics (23). It is proposed that most of these potential applications of low-dose naltrexone (<1 mg oral dose) can be improved by substituting LD-6BN for naltrexone.

Low-dose naltrexone (0.001-1.0 mg oral dose) has been shown to have positive outcomes in diverse pathophysiological states (review: (23)), some of these applications parallel those outlined for 6β-naltrexol here, but naltrexone potently causes withdrawal or aversive symptoms because of its inverse agonist effect when increased spontaneous MOR-μ* signaling is present. Thereby, naltrexone non-competitively blocks both opioid analgesia and causes withdrawal with equally high potency, in contrast to the much lower 6BN potency in these effects (requires competitive inhibition of opioid analgesic activation of MOR-μ to MOR-μ* (13). While both 6β-naltrexol and naltrexone have nearly equally robust affinities for MOR, in guinea pigs and rhesus monkeys, 6β-naltrexol is >100-fold less potent than naltrexone in blocking central opioid effect (analgesia) or causing withdrawal in dependent animals (13,14). In opioid weaning-detoxification protocols, addition of low-dose naltrexone during opioid detoxification/weaning with descending doses of buprenorphine is limited initially to 0.25 mg naltrexone total oral dose; any higher dose will cause withdrawal symptoms (24-25). As 6β-naltrexol is the main metabolite of naltrexone and accumulates to higher levels than naltrexone in the blood after naltrexone dosing, the newly established high potency of 6β-naltrexol as an addiction modulator indicates that low-dose naltrexone's clinical effects are indeed mediated by 6β-naltrexol as naltrexone's metabolite. Further applications of low-dose naltrexone include anti-inflammatory treatments of chronic pain associated with multiple sclerosis, fibromyalgia, and more (26,27). In a specific application, neuropathic chronic pain, allodynia, and hyperalgesia appear to be correlated with the status in peripheral sensory neurons, and afferent nociceptors, where MOR appears to exist largely in a silent state, non-responsive to opioid agonists (termed here MOR-μ^{x}) (4,17-19).

Upon inflammatory stimuli, MOR can get activated and establishes a level of spontaneous signaling via MOR-μ*, reducing pain sensation. However, continued stimulation of these peripheral MOR sites leads to elevated lasting MOR-μ* signaling, which sensitizes nociceptors and leads to hyperalgesia (17,18), with diminishing efficacy of opioid analgesics. In addition, naltrexone appears to lock MOR into the active MOR-μ* state while blocking MOR-μ* signaling, thereby, preserving the dependent state upon washout, whereas 6BN reverses MOR-μ* back to the inactive state, demonstrated in peripheral sensory neurons after washing out the drugs (4). It is perhaps indicative of the state of the art that the concentration of 6BN tested in vitro was 10 μM (4)-˜10,000-fold higher than the expected EC50 of 6BN invoked in this invention. Therefore, replacing low-dose naltrexone with 6β-naltrexol dosing can lead to superior clinical outcomes not limited by naltrexone's side effects as an inverse agonist. Clinical uses proposed for low-dose naltrexone (0.001-3.0 mg) (23) are inherently included in this invention. In another previous implementation, ultra-low dose naltrexone (0.001 mg or less oral dose) has been suggested to improve opioid pain management (31), by as yet uncertain mechanisms, but clinical implementation is lacking; we do not consider this dosage range to be robustly effective for 6BN. LD-6BN is designed to selectively suppress key elements of addiction at doses that do not interfere with opioid analgesia nor cause withdrawal in opioid-dependent subjects. It was shown that the ID50 of i.v. 6BN is ˜3 mg against 10 mg morphine induced slowing of bowel movements in opioid naïve subjects (28), whereas 20 mg 6BN did not suppress analgesia (highest dose tested; 6BN was administered 30 min before morphine), demonstrating its peripheral selectivity at higher doses. In a separate pilot study, highly opioid-dependent methadone maintenance subjects (n=4) tolerated 0.5-1.0 mg 6BN given i.v. while experiencing peripheral effects and bowel movements, but no central withdrawal symptoms (20,21). One can expect that this action is caused by 6BN antagonism at peripheral MOR, as opioid-dependent subjects are highly sensitive to any antagonist action. Therefore, in the context of the clinical application targeted here, and assuming 30% bioavailability of oral 6BN, the upper limit of an oral LD-6BN dosage regimen (assuming ˜30% bioavailability) is 1-3 mg if designed to act solely as an addiction modulator.

Since low dose naltrexone is effective in facilitating opioid weaning (23-25), an action likely mediated at least in part by its metabolite 6BN, the LD-6BN dose range for, for example but not limited to, oral application to be in the range of 0.001 to 3.0 mg 6BN. In one embodiment, the dose of 6BN is from 0.001 to 0.25 mg. In another embodiment, the dose of 6BN is from 0.25 to 0.50 mg. In a further embodiment, the dose of 6BN is from 0.50 to 0.75 mg. In a still further embodiment, the dose of 6BN is from 0.75 to 1.0 mg. In a still another embodiment, the dose of 6BN is from 1.0 to 1.25 mg. In another embodiment, the dose of 6BN is from 1.25 to 1.5 mg. In a further embodiment, the dose of 6BN is from 1.5 to 1.75 mg. In a still further embodiment, the dose of 6BN is from 1.75 to 2.0 mg. In a still further embodiment, the dose of 6BN is from 2.0 to 3.0 mg. These projected dose ranges can be modified once clinical results with LD-6BN are available (for example oral bioavailability, estimated here to be ˜30% as observed in rodents.

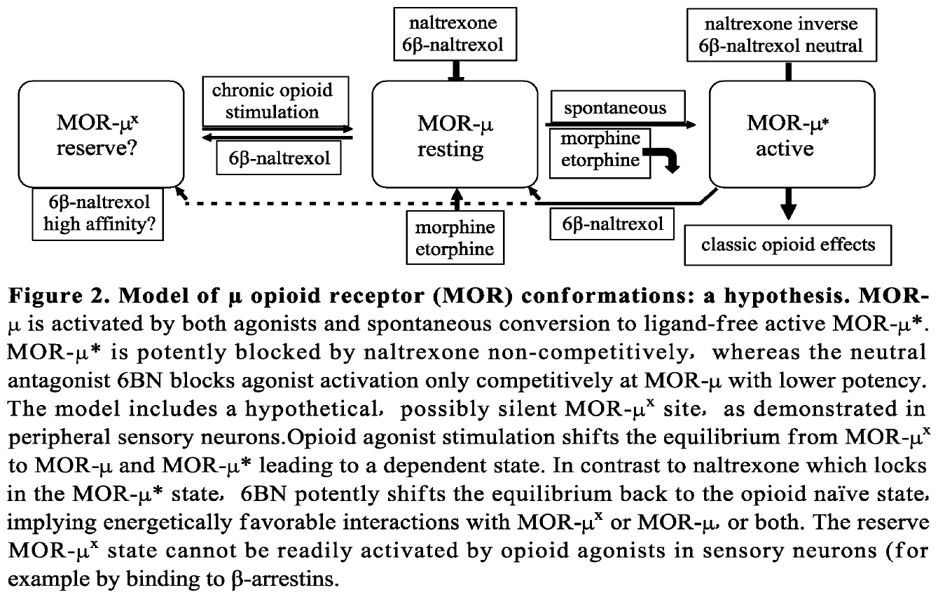

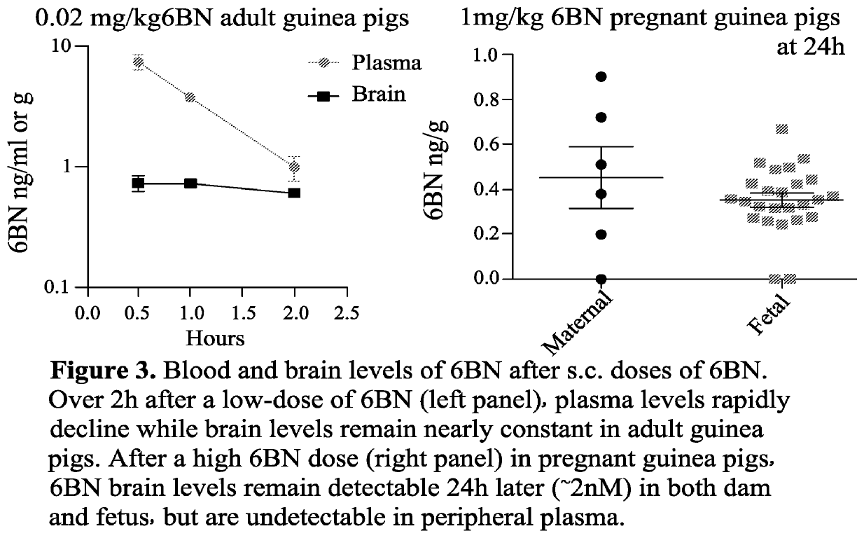

In view of the proposed dynamics between MOR conformations between dependent and non-dependent states, influenced by low but persistent occupancy after LD-6BN dosing, one can expect single daily dosing to be effective even when used together with long-acting opioid such as methadone. This prediction does not preclude application in slow-release formulations, or co-formulation with an opioid analgesic in a combined pharmaceutical preparation. 6β-Naltrexol has already shown extreme potency in preventing the development of opioid dependence in animal models, without interfering with opioid analgesia mediated at MOR-μ*. To account for its high potency in reversing the opioid dependent state, characterized by high MOR-μ* activity, the model of MOR conformations shown in FIG. 2 is proposed, adapted from Sadee et al. (16). Whereas opioid agonists shift the equilibrium to the right and gradually impose a sustained high level of active MOR-μ* in the dependent state, 6BN shifts the equilibrium back to the left (MOR-μ and MOR-μ^{x}). MOR-μ^{x} is likely to exist in several conformations, for example a labile unusual MOR sub-population in rodent brain, labeled with ³H-naloxone and accounting for ˜40% of all ³H-naloxone binding sites (11), or a silent form bound to β-arrestins as shown in sensory neurons (4), the latter endowed with alternative signaling functions on its own via β-arrestins (differentially activated by biased agonists) (9,10,43). MOR-μ^{x} can be considered either a receptor reserve of MOR, or an alternative functional form, or both. The schematics in FIG. 2 stipulates that 6BN can affect MOR signaling by two mechanisms, one through competitive antagonism (low potency) of agonists activating MOR-μ to MOR-μ*, and the other by gradually converting MOR-μ* back to its resting MOR-μ or MOR-μ^{x} reserve states (high potency, requiring only low receptor occupancy over extended time periods), or both. This proposition is supported by our finding that 6BN becomes more potent as an antagonist at higher dose levels when administered to mice ˜2 h before a dose of morphine, compared to administration at the same time (45,47, U.S. Pat. No. 8,748,448). Similarly, 6β-naltrexamide—also a peripherally selective neutral opioid antagonist with high MOR affinity—gains potency when preinjected before morphine (see US patent U.S. Pat. No. 8,748,448). At the time of filing these patents, the mechanism underlying the delayed action was unknown.

Whereas 6BN penetrates the blood-brain-barrier slowly, accounting for its peripheral selectivity at higher doses when designed to act as a neutral opioid antagonist, at low doses 6BN is selectively retained in the brain, mostly residing at its own receptor sites, and thus reaches higher levels in the brain than in blood 2-3 h after dosing in animals, and is retained in the brain for prolonged time period (FIG. 3). Therefore, when serving as an addiction modulator, LD-6BN interacts effectively with both peripheral and central opioid receptors. Its effect on reversing opioid induced hyperalgesia might at least in part occur by acting on MOR in peripheral nociceptor neurons (15), but reversal of opioid dependence is likely driven by a central effect. The level of MOR bound 6BN after an s.c. dose of 0.02 mg/kg is estimated to occupy ˜10% of MOR sites, assuring a gradual reversal of MOR-μ* to MOR-μ and MOR-μ^{x}, insufficient to interfere with opioid analgesia, or cause withdrawal in dependent subjects.

The unique relationships between opioid receptor conformations and 6BN, represented in a novel receptor model (FIG. 2), enable discovery of novel opioid addiction modulators including 6BN analogs (patents (47). An effective potent addiction modulator must have the following pharmacological characteristics:
1. High affinity at MOR (measured with in vitro binding studies that however do not reflect MOR conformations in vivo)
2. High affinity at MOR in vivo with sufficient strength to maintain sustained brain levels via retention at MOR sites (for example 6BN maintains 0.5 ng/g brain 24 h after a 1 mg/kg s.c. dose to guinea pigs even though the initial blood t1/2 is ˜1 h) (FIG. 3)); such long retention is accounted for by a receptor micro-compartment effect for potent antagonists (44).
3. Neutral antagonism at ligand-free MOR-μ*, binding with high affinity while not interfering with opioid signaling; essential to endow only moderate potency as a MOR antagonist, which requires competitive binding with the agonist at MOR-μ to prevent activation to begin with.
4. High potency in facilitating gradual conversion of the dependent state (elevated MOR-μ*) back to the resting and/or reserve MOR states. Following LD-6B dosing, only a low fractional receptor occupancy is sufficient to reverse the dependent state in a gradual dynamic fashion, further accounting for high potency as an addiction modulator.
5. When designed to act as an antagonist (doses above the LD-6BN range), potency is expected to increase over time when administered before an opioid agonist, presumably via high MOR-μ* occupancy and thereby more rapid reversal of MOR-μ* back to its resting or reserve states. Thus, at high 6BN doses, and high MOR occupancy, this process reduces opioid induced analgesia with a delayed action (maximally ˜2 h pre-injection time for 6BN in mice (45, and patents (47)); however, at very low doses (e.g. 0.02 mg/kg in animals) MOR-μ* occupancy is estimated to be only in the order of 10%, slowing the reversal of the MOR-μ* state in such a way that analgesia is unaffected (FIG. 3).
6. Peripheral selectivity may be a useful attribute to bolster suppression of opioid induced hyperalgesia, mediated at least in part peripherally (15,17-19) or for treatment of opioid induced bowel dysfunction (requiring intermediate dose ranges), also a peripheral effect; these applications for LD-6BN are further supported by long half-lives of an addiction modulator in blood (˜12 h for 6BN in humans (45)).
7. Morphinan analogues of 6BN tend to bind to all opioid receptors with varying potency and efficacy (patents (47)). For example, the inventor found that 6BN has detectable inverse agonist activity at kappa opioid receptors (KOR) pretreated in vitro with KOR agonists (46). Potency of potential addiction modulators should therefore be tested for binding and efficacy at MOR, DOR (delta), and KOR, to detect differences between them and anticipate potential variation in clinical outcomes.

A person of ordinary skill in the art will be able to combine these characteristics and the methods described in the references to search for novel addiction modulators with actions similar to those of 6BN.

The invention includes opioid antagonists specified in U.S. Pat. No. 6,713,488 B2, U.S. Pat. Nos. 8,748,448, 8,883,817B2, 9,061,024B2, and EP2214672, all of which are expressly incorporated herein by reference. Also included are general screening assays for chemical compounds having similar characteristics. Relative receptor activity at MOR-μ-MOR-μ* can be determined in MOR-transfected cells (46), or with ³H- and ¹¹C-labeled 6beta-naltrexol in cell culture or in vivo in animal models using binding assays (11,44), or PET scans. A key characteristic of an addiction modulator is the increased potency as an antagonist when administered before the agonist (45), accounting for a delayed action as predicted by the MOR model (FIG. 2). Assays for measuring basal MOR signaling and antagonist effects have been published (1-3). Compounds can be further tested in vivo for ability at low doses to prevent opioid dependence (8) and hyperalgesia (15), or treat other drug use disorders, such as alcoholism as shown with naltrexone (38,39).

Taken together, results indicate that low-dose 6β-naltrexol will be effective in modulating the dependent state of the opioid receptor system, thereby reducing compulsory alcohol consumption in AUD, and in other conditions with upregulated, dysregulated basal MOR signaling.

While 6β-naltrexol is one compound that is useful for the treatment protocol claimed herein, other naltrexol and naloxol derivatives, analogs, and/or metabolites, such as those specified in U.S. Pat. No. 8,748,448, U.S. Ser. No. 14/278,576, European Patent No. EP2214672 and U.S. Ser. No. 12/288,347 (all of which are expressly incorporated by reference, including routes of administration and sustained release formulations), also have neutral antagonist properties and the same structure as 6β-naltrexol that is recognized by export pumps in the blood-brain barrier, indicating they can also be peripherally selective. The invention embodies usage of any of these compounds or derivatives or metabolites thereof. For example, 6α-naltrexol, 6α- and β-naloxol and their derivatives (including PEGylated derivatives), and 6α/β-naltrexamine and 6α/β-naloxamine, and specifically 6β-naltrexamide, all show neutral antagonism. 6β-Natrexamide has also been shown to have strong peripherally selective activity (internal results, and patents (37)).

### Pharmaceutical Compositions

Pharmaceutical compositions, such as co-formulation with an opioid analgesic, and single unit dosage forms comprising a compound of the invention, or a pharmaceutically acceptable prodrug, salt, solvate or hydrate thereof, are also encompassed by the invention and methods of use disclosed herein. Individual dosage forms of the invention may be suitable for oral, mucosal (including sublingual, buccal, rectal, nasal, or vaginal), parenteral (including subcutaneous, intramuscular, bolus injection, intraarterial, or intravenous), transdermal, or topical administration.

Pharmaceutical compositions and dosage forms of the invention comprise a compound of the invention, or a pharmaceutically acceptable prodrug, salt, solvate or hydrate thereof. Pharmaceutical compositions and dosage forms of the invention typically also comprise one or more pharmaceutically acceptable excipients.

A particular pharmaceutical composition encompassed by this embodiment comprises a compound of the invention, or a pharmaceutically acceptable prodrug, salt, solvate or hydrate thereof, and at least one additional therapeutic agent. Examples of additional therapeutic agents include, but are not limited to opioid analgesics, immune suppressor agents, anti-cancer drugs and anti-inflammation therapies.

Single unit dosage forms of the invention are suitable for oral, mucosal (e.g., nasal, inhalation, sublingual, vaginal, buccal, or rectal), parenteral (e.g., subcutaneous, intravenous, bolus injection, infusion, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of inflammation or a related disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton PA (1990).

Typical pharmaceutical compositions and dosage forms comprise one or more carriers, excipients or diluents. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms, and topical absorption enhancers form use in transdermal applications. The suitability of a particular excipient may also depend on the specific active ingredients in the dosage form.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, NY, 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

In one embodiment, the compound of the invention is administered in a pharmaceutical formulation comprising carbonate.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

In one embodiment, the compounds of the invention are administered in a pharmaceutical composition comprising liposomes. The liposomes may be polymerized or unpolymerized and the compound of the invention may optionally be intercalated within the liposomes. Suitable lipophilic solvents or vehicles include fatty oils such as sesame oil, or synthetic fatty acid esters, such as ethyl oleate or triglycerides.

### Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein, such as organic solvents including propylene glycol, polyethylene glycol, ethanol, glycerol, polyethylene glycol ricinoleate (Cremophor) or polyoxyethylene sorbitan fatty acid esters (Tween), can also be incorporated into the parenteral dosage forms of the invention. Parenteral solutions of the compounds of the invention can also comprise human serum proteins which serve as crystallization inhibitors, such as those described in U.S. Pat. No. 4,842,856, incorporated by reference herein in its entirety. Parenteral solutions of the compounds of the invention can further comprise poloxamers or polysorbates.

Parenteral dosage forms can also be administered in depot, long acting or slow-release forms comprising a compound of the invention in a matrix of a polymer of polyols and hydroxy carboxylic acids such as those disclosed in International Publication WO 78/00011, incorporated herein by reference in its entirety. Depot forms can also comprise a polyol ester containing polymeric-dicarboxylic acid residues (e.g. tartaric acid) such as those described in U.S. Pat. Nos. 5,922,682 and 5,922,338, each of which is incorporated herein by reference in its entirety. Additional depot forms include matrices comprised of an ester of polyvinyl alcohol (M.W. of about 14000), polyethylene glycol (M.W. of about 6000 to 20,000) or polymer hydroxycarboxylic ester residues (e.g., lactic acid M.W. of about 26,000 to 114,000) or glycolic acid (M.W. of about 10,000), such as those disclosed in European application No. 92918, incorporated herein by reference in its entirety. Delayed release formulations for parenteral dosage forms also include binder-free granules as disclosed in U.S. Pat. No. 4,902,516 and those disclosed for use with vitamin D in U.S. Pat. No. 5,795,882, each incorporated by reference herein in its entirety.

Further parenteral dosage forms include wax microspheres such as those disclosed in U.S. Pat. No. 6,340,671, lipophilic formulations such as those disclosed in U.S. Pat. No. 6,335,346, non-aqueous compositions such as those disclosed in U.S. Pat. No. 5,965,603, carbohydrate polymers such as those disclosed in U.S. Pat. No. 5,456,922 and emulsions such as those disclosed in U.S. Pat. Nos. 4,563,354 and 5,244,925, each incorporated by reference herein in its entirety.

Parenteral dosages can be delivered via implantable devices, osmotic pumps, or catheter systems which are capable of delivering the composition at selectable rates (See U.S. Pat. Nos. 6,471,688; 6,436,091; 6,413,239; 6,464,688; 5,672,167; and 4,968,507, each incorporated by reference herein in its entirety).

### Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton PA (1990) or Remington: The Science and Practice of Pharmacy, 20th ed., Lippincott, Williams and Wilkins, (2000).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Oral dosage forms containing excipients developed to enhance paracellular transport and to reduce enzymatic degradation in the gastrointestinal tract are also useful to increase oral bioavailability of compounds of the invention. One such technology is described by Maher S, et al. (2016) Drug Deliv Rev.106(Pt B):277-319. Another such technology is described by Mehta N. et al (Mehta, Nozer & Stern, William & Carl, Stephen & Vrettos, John & Sturmer, Amy. (2013) Biopolymers, 23rd American Peptide Symposium, 100: 237.

Oral Delivery with PEPTELLIGENCE™: Examples of Preclinical and Clinical Studies with Peptides. 237-237.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, PA), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103□ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, MD), a coagulated aerosol of synthetic silica (marketed by Degussa Co. of Plano, TX), CAB-O-SIL (a pyrogenic silicon dioxide product sold by Cabot Co. of Boston, MA), and mixtures thereof. If used at all, lubricants are typically used in an amount of less than about 1 weight percent of the pharmaceutical compositions or dosage forms into which they are incorporated.

### Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, carboxymethyl cellulose, or other polymer matrices, gels, permeable membranes, osmotic systems, multilayer coatings, microparticles, liposomes, microspheres, or a combination thereof to provide the desired release profile in varying proportions. In a preferred embodiment, the controlled-release formulation is biodegradable. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled release. The compound of the invention may also be administered in a depot formulation or inclusion complex and can optionally be inserted under the skin.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

### Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 18th eds., Mack Publishing, Easton PA (1990); and Introduction to Pharmaceutical Dosage Forms, 4th ed., Lea & Febiger, Philadelphia (1985). Dosage forms suitable for treating mucosal tissues within the oral or nasal cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 18th eds., Mack Publishing, Easton PA (1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to, acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

The invention will now be further described in the Examples below, which are intended as an illustration only and do not limit the scope of the invention.

### EXAMPLES

### Example 1

### Use of LD-6BN to Enhance Efficacy and Reduce Adverse Effects of Narcotic Analgesics in the Treatment of Pain

Since LD-6BN appears to act in a non-competitive fashion, its potency in preventing opioid dependence and hyperalgesia is largely independent of the specific opioid analgesic and its dose. Therefore, the estimated dosage regimen when given concomitantly with opioid analgesics is captured by the range defined for LD-6BN: 0.001-3 mg orally given once or twice daily. 6BN doses above 3 mg may be needed to suppress peripheral side effects such as opioid induced constipation, but such doses may cause some peripheral withdrawal symptoms such as bowel movement, when given the first time to opioid dependent patients, and doses may need to be adjusted relative to the opioid analgesics doses, as 6BN then acts as a competitive antagonist.

LD-6BN is expected to be sufficient to alleviate opioid induced bowel dysfunction more generally if related to enhanced prolonged MOR-μ* activity. It is also expected to reduce drug seeking behavior if driven by MOR-μ* activity.

LD-6BN (0.001-3 mg orally once or twice daily) has the capacity to alleviate long-term adverse effects of opioid maintenance therapies (methadone and buprenorphine (with or without low-dose naloxone which has limited bioavailability and short half-life)), including dependence, hyperalgesia, bone loss, and more.

LD-6BN (0.001-3 mg orally once or twice daily) will reduce withdrawal symptoms in opioid dependent subjects, by reducing dependence. A preferred dosage regimen to facilitate weaning involves daily LD-6BN administration for 7 days before weaning begins, and continued daily LD-6BN during the weaning procedure (e.g., decreasing doses of an opioid analgesic (including opioid maintenance opioids over one week), and continued dosing after completed weaning. Standard methods to prevent relapse will then be implemented.

Dosage regimen for LD-6BN designed to alleviate diverse conditions related to endogenous opioid peptide activity and signaling (without concomitant application of opioid analgesics) can use the same dosage range defined for LD-6BN. These conditions include but are not limited to compulsive addictive behaviors including drug addiction, eating disorders, excessive gambling, and more. Drug addiction involves alcohol, opioids, nicotine, cocaine, other stimulants, and more. Following weaning for opioid addiction, dependence and craving remain high for some time, so that LD-6BN can serve to reduce these aspects of opioid use disorders after discontinuation of opioid drugs. In addition, elevated basal MOR-μ* activity appears to be involved in neuropathic pain, allodynia, and hyperalgesia, inflammation and immune related dysregulation and pain, and conditions where low dose naltrexone (0.001-3.0 mg oral dose) have shown some efficacy (review: (23). Further applications of low-dose naltrexone include anti-inflammatory treatments of chronic pain associated with multiple sclerosis, fibromyalgia, and more (26,27). In view of the potential advantages of LD-6BN over low dose naltrexone, one embodiment of this invention is to replace naltrexone (or similar inverse opioid antagonists such as nalmefene) with LD-6BN to achieve superior outcomes.

LD-6BN can also be effective in preventing neonatal opioid withdrawal syndrome (NOWS) without the need to escalate 6BN doses—complementing an existing patent on preventing NOWS by 6BN therapy prenatally to the mother.

### Example 2

### LD-6BN Dosage Formulations

As 6BN is highly water soluble, has oral bioavailability of ˜30% in humans, and a long duration of action, oral dosing with conventional tablets or capsules is a primary embodiment. Additional preferred formulations include transdermal patch application and intranasal application (with partial direct access to the brain, bypassing the blood-brain-barrier). When combined with opioid analgesics in a co-formulation, LD-6BN can be delivered in the same formulation type for any of the opioid analgesics currently used clinically, including sustained release oral preparations. No dosage adjustments are needed.

### Example 3

Analogs and derivatives of 6BN suitable for the intended applications Given the defined criteria for the class of addition modulators with 6BN as the lead example, the naltrexol and naloxol analogs identified in the patents cited herein, including but not limited to U.S. Pat. No. 6,713,488 B2, U.S. Pat. Nos. 8,748,448, 8,883,817B2, 9,061,024B2, and EP2214672, all of which are expressly incorporated herein by reference, meet one or more of these criteria and are therefore incorporated here—while the proposed methodology can reveal novel chemical entities as drug candidates. For example, analogs and derivatives can include 6β-naltrexamide, 6α-naltrexol, 6α-naloxol, 6β-naloxol, 6α-naltrexamine, 6β-naltrexamine, 6α-naloxamine, or 6β-naloxamine, or C-6 derivatives thereof, such as carboxyl esters, amides, or ethers (including pegylated derivatives), or reduced naltrexol or naloxol with a —C-6H₂ substitution. A specific drug candidate meeting several required criteria already tested is 6β-naltrexamide (included in previously cited patents as a neutral peripherally selective MOR antagonist): high affinity to MOR in vitro, low potency as a neutral antagonist, increasing potency as a neutral antagonist in mice when given 2 h before the opioid analgesic morphine, peripheral selectivity. It is proposed that low-dose 6β-naltrexamide is a prime drug candidate acting as an addiction modulator.

### REFERENCES

1. Sadee, W, Wang, D, and Bilsky, E J (2005) Basal opioid receptor activity, neutral antagonists, and therapeutic opportunities. Life Sci 76, 1427-1437.
2. Raehal K M, Lowery J J, Bhamidipati C M, Paolino R M, Blair J R, Wang D, Sadée W, Bilsky E J. (2005). In vivo characterization of 6beta-naltrexol, an opioid ligand with less inverse agonist activity compared with naltrexone and naloxone in opioid-dependent mice. J Pharmacol Exp Ther. 313, 1150-1162.
3. Sirohi S, Dighe S V, Madia P A, Yoburn B C (2009) The relative potency of inverse opioid agonists and a neutral opioid antagonist in precipitated withdrawal and antagonism of analgesia and toxicity. J Pharmacol Exp Ther. 330, 513-519.
4. Laura C. Sullivan, Teresa S. Chavera, Raehannah J. Jamshidi, Kelly A. Berg, and William P. Clarke. Constitutive Desensitization of Opioid Receptors in Peripheral Sensory Neurons. J Pharmacol Exp Ther 359:411-419, December 2016. http://dx.doi.org/10.1124/jpet.116.232835
5. Walwyn W M, Chen C, Kim H, Minasyan A, Ennes H S, McRoberts J A, Marvizon J C G. Sustained suppression of hyperalgesia during latent sensitization by _mu-, _delta-, and _kappa-opioid receptors and _2A-adrenergic receptors: role of constitutive activity. Neurobiol. Dis. 36:204-221 (2016).
6. Wang D, Raehal K M, Lin E T, Lowery J J, Kieffer B L, Bilsky E J, Sadée W (2004) Basal signaling mu opioid receptor in mouse brain: role in narcotic dependence. J Pharm Exp Ther 308, 512-520
7. D. Wang, K. M. Raehal, E. J. Bilsky, W. Sadée. Inverse agonists and neutral antagonists at μ opioid receptor (MOR): Possible role of basal receptor signaling in narcotic dependence. J.Neurochem., 77: 1590-1600 (2001)
8. J. Oberdick, Y. Ling, M. A. Phelps, M. S. Yudovich, K. Schilling, W. Sadee. Preferential delivery of an opioid antagonist to the fetal brain in pregnant mice. J.Pharmacol.Exp.Ther. 358: 22-30 (2016). PMID:27189967
9. Zhu, X., Finlay, D. B., Glass, M. et al. An intact model for quantifying functional selectivity. Sci Rep 9, 2557 (2019) doi:10.1038/s41598-019-39000-z
10. Grim T W, Schmid C L, Stahl E L, Pantouli F, Ho J H, Acevedo-Canabal A, Kennedy N M, Cameron M D, Bannister T D, Bohn L M. A G protein signaling-biased agonist at theμ-opioid receptor reverses morphine tolerance while preventing morphine withdrawal. Neuropsychopharmacology. 2019 Aug. 23. doi: 10.1038/s41386-019-0491-8. [Epub ahead of print] PMID:31443104
11. J. Grevel and W. Sadée. An Opiate Binding Site in Rat Brain is Highly Selective for 4,5-Epoxymorphinans. Science 221, 1198-1201 (1983).
12. J. M. Quillan, K. W. Carlson, C. Song, D. Wang, and W. Sadée. Differential Effects of μ Opioid Receptor (MOR) Ligands on Ca²⁺ Signaling. J.Pharmacol.Exp.Ther., 302: 1002-1012 (2002).
13. Porter S J, Somogyi A A, White J M. (2002) In vivo and in vitro potency studies of 6beta-naltrexol, the major human metabolite of naltrexone. Addict Biol. 7, 219-225.
14. Ko M C, Divin M F, Lee H, Woods J H, Traynor J R. (2006) Differential in vivo potencies of naltrexone and 6beta-naltrexol in the monkey. J Pharmacol Exp Ther. 316, 772-779.
15. Corder G, Tawfik V L, Wang D, Sypek E I, Low S A, Dickinson J R, Sotoudeh C, Clark J D, Barres B A, Bohlen C J, Scherrer (2017). Loss of μ opioid receptor signaling in nociceptors, but not microglia, abrogates morphine tolerance without disrupting analgesia. Nat Med. 23:164-173.
16. W. Sadee, J. Oberdick, Z. Wang. Biased Opioid Antagonists as Modulators of Opioid Dependence: Opportunities to Improve Pain Therapy and Opioid Use Management.). Molecules 25, 4163; doi:10.3390/molecules25184163 (2020). Same as (22,29)
17. Corder G, Doolen S, Donahue R R, Winter M, Jutras B, He Y, Hu X, Wieskopf J S, Mogil J S, Storm D R, Wang Z J, McCarson K E, and Taylor B K, Constitutive μ-opioid receptor activity leads to long-term endogenous analgesia and dependence. Science 2013; 341,1394-1399. doi:10.1038/nm.4262
18. Araldi D, Ferrari L F, Levine J D. Hyperalgesic priming (type II) induced by repeated opioid exposure: maintenance mechanisms. Pain 158:1204-1216 (2017). PMID:28306605
19. Araldi D, Ferrari L F, Levine J D D. J Neurosci. 2015 Sep. 9; 35(36):12502-17. doi: 10.1523/JNEUROSCI.1673-15.2015. Repeated Mu-Opioid Exposure Induces a Novel Form of the Hyperalgesic Priming Model for Transition to Chronic Pain. PMID:26354917
20. Mendelson J, Li L, Flower K, Harris W, Coyle J R, et al. (2011) The Effects of 6β-Naltrexol, a Putative Neutral Opioid Antagonist, in Opioid-Dependent Subjects: A Proof-of-Concept Trial. J Addict Res Ther 2:108. doi:10.4172/2155-6105.1000108
21. AIKO Biotechnology. A Phase-I, Two-Stage, Double-Blind, Placebo-Controlled, Pharmacokinetic and Pharmacodynamic Trial of Low Doses of Intravenous 6β-Naltrexol (AIKO-150) in Opioid-Dependent Subjects. ClinicalTrials.gov. January 2009. https://clinicaltrials.gov/ct2/show/NCT00829777?term=AIKO&rank=1>.
23. Low-Dose Naltrexone (LDN)-Review of Therapeutic Utilization.
   Toljan K, Vrooman B.Med Sci (Basel). 2018 Sep. 21; 6(4):82. doi: 10.3390/medsci6040082.PMID: 30248938
24. Withdrawal severity and early response to treatment in the outpatient transition from opioid use to extended release naltrexone.
   Mannelli P, Swartz M, Wu L T. Am J Addict. 2018 September;27(6):471-476. doi: 10.1111/ajad.12763. Epub 2018 Jul. 20.PMID: 30028058
25. Mannelli, P.; Patkar, A. A.; Peindl, K.; Gorelick, D. A.; Wu, L.-T.; Gottheil, E. Very low dose naltrexone addition in opioid detoxification: A randomized, controlled trial. Addict. Biol. 2009, 14, 204-213. [CrossRef] [PubMed]
26. The use of low-dose naltrexone (LDN) as a novel anti-inflammatory treatment for chronic pain. Younger J, Parkitny L, McLain D.Clin Rheumatol. 2014 April; 33(4):451-9. doi: 10.1007/s10067-014-2517-2. Epub 2014 Feb. 15.PMID: 24526250
27. The Safety and Efficacy of Low-Dose Naltrexone in the Management of Chronic Pain and Inflammation in Multiple Sclerosis, Fibromyalgia, Crohn's Disease, and Other Chronic Pain Disorders. Patten D K, Schultz B G, Berlau D J.Pharmacotherapy. 2018 March;38(3):382-389. doi: 10.1002/phar.2086. Epub 2018 Feb. 23.PMID: 29377216
28. Yancey-Wrona, J, Dallaire, B, Bilsky, E, Bath, B, Burkart, J, Wenster, L, Magiera, D, Yang, X, Phelps, M, and Sadee, W. (2011) 6β-naltrexol, a peripherally selective opioid antagonist that inhibits morphine induced slowing of gastrointestinal transit: an exploratory study. Pain Medicine 12, 1727-1737.
30. A. Safa, A. R. Lau, S., Aten, K. Schilling, K. L. Bales, V. Miller, J. Fitzgerald, M. Chen, K. Hill, K. Dzwigalski, K. Obrietan, M. A. Phelps, W. Sadee, J. Oberdick. Pharmacological prevention of neonatal opioid withdrawal in a pregnant guinea pig model. bioRxiv, 2020 preprint mdpi. doi: https://doi.org/10.1101/2020.07.25.221192. Front.Pharmacology, DOI: 10.3389/fphar.2020.613328 (2021)
31. Webster, L. R.; Butera, P. G.; Moran, L. V.; Wu, N.; Burns, L. H.; Friedmann, N. Oxytrex Minimizes Physical Dependence While Providing Effective Analgesia: A Randomized Controlled Trial in Low Back Pain. J. Pain 2006, 7, 937-946. [CrossRef] [PubMed]
32. Noncanonical scaffolding of Gai and D-arrestin by G protein-coupled receptors. Smith J S, Pack T F, Inoue A, Lee C, Zheng K, Choi I, Eiger D S, Warman A, Xiong X, Ma Z, Viswanathan G, Levitan I M, Rochelle L K, Staus D P, Snyder J C, Kahsai A W, Caron M G, Rajagopal S.Science. 2021 Mar. 12; 371(6534):eaay1833. doi: 10.1126/science.aay1833. Epub 2021 Jan. 21.PMID: 33479120
33. Influence of opioids on immune function in patients with cancer pain: from bench to bedside. Boland J W, Pockley A G. Br J Pharmacol. 2018 July;175(14):2726-2736. doi: 10.1111/bph.13903. Epub 2017 Jul. 23.PMID: 28593737
34. [Pain inhibition by opioids-new concepts].
   Stein C.Anaesthesist. 2019 February;68(2):97-103. doi: 10.1007/s00101-018-0528-0.PMID: 30645691
35. Pain and sickle cell disease.

### Aich A, Jones M K, Gupta K.Curr Opin Hematol. 2019 May; 26(3):131-138. doi: 10.1097/MOH.0000000000000491.PMID: 30893088

36. American Society of Hematology 2020 guidelines for sickle cell disease: management of acute and chronic pain.
   Brandow A M, Carroll C P, Creary S, Edwards-Elliott R, Glassberg J, Hurley R W, Kutlar A, Seisa M, Stinson J, Strouse J J, Yusuf F, Zempsky W, Lang E. Blood Adv. 2020 Jun. 23; 4(12):2656-2701. doi: 10.1182/bloodadvances.2020001851.PMID: 32559294
37. Mu opioid receptor: a gateway to drug addiction.
   Contet C, Kieffer B L, Befort K. Curr Opin Neurobiol. 2004 June;14(3):370-8. doi: 10.1016/j.conb.2004.05.005.PMID: 15194118
38. Bi-directional Acceleration of Alcohol Use and Opioid Use Disorder.

### Huang W, Penaherrera E P, Desir D F, Gamarro D L, Cottrell J, Chu T, Chang S L. J Drug Alcohol Res. 2019 Oct. 18; 2019:236084.PMID: 32440365

39. Pharmacotherapy for adults with alcohol use disorders in outpatient settings: a systematic review and meta-analysis.
   Jonas D E, Amick H R, Feltner C, Bobashev G, Thomas K, Wines R, Kim M M, Shanahan E, Gass C E, Rowe C J, Garbutt J C. JAMA. 2014 May 14;311(18):1889-900. doi: 10.1001/jama.2014.3628.PMID: 24825644
40. Naltrexone: A Pan-Addiction Treatment?
   Aboujaoude E, Salame W O. CNS Drugs. 2016 August; 30(8):719-33. doi: 10.1007/s40263-016-0373-0.PMID: 27401883
42. Obsessive-compulsive disorder, impulse control disorders and drug addiction: common features and potential treatments.

### Fontenelle L F, Oostermeijer S, Harrison B J, Pantelis C, Yücel M.Drugs. 2011 May 7;71(7):827-40. doi: 10.2165/11591790-000000000-00000.PMID: 21568361

43. J. S. Smith et al., Noncanonical scaffolding of Gₐᵢ and b-arrestin by G protein-coupled receptors. Science 371, eaay1833 (2021). DOI: 10.1126/science.aay1833
44. Opiate Antagonist Receptor Binding In Vivo: Evidence for a New Receptor Binding Model. D. C. Perry, K. B. Mullis, S. Øie and W. Sadée. Brain Research 199, 49-61 (1980)
45. 6β-Naltrexol preferentially antagonizes opioid effects on gastrointestinal transit compared to antinociception in mice. J. Yancey-Wrona, T. J. Raymond, H. K Mercer, W. Sadee, E. J. Bilsky Life Sci., 413-420 (2009)
46. Different effects of opioid antagonists on mu, delta, and kappa opioid receptors with and without agonist pretreatment. D. Wang, X. Sun, W. Sadee. J.Pharmacol.Exp.Ther 321: 544-552 (2007).

It is to be understood that the invention is not limited to the particular embodiments of the invention described above, as variations of the particular embodiments may be made and still fall within the scope of the appended claims.

What is claimed is:

1. A method for the treatment of a condition involving upregulated basal signaling of the μ-opioid receptor (MOR) system, comprising the step of administering 0.001 to 3.0 mg of 6β-naltrexol, or a derivative or analog thereof, to an individual in need thereof.

2. The method according to claim 1, wherein said condition is opioid dependence, hyperalgesia, chronic neuropathic pain, alcoholism, nicotine addiction, cocaine and stimulant abuse, compulsive behaviors, anorexia, binge eating, gambling or excessive sexual behaviors.

3. The method according to claim 1, wherein 6β-naltrexol is administered at a dosage insufficient to block conventional opioid agonist effects but sufficient gradually to bind and deplete the MOR-μ* site.

4. The method according to claim 1, wherein the dose of 6β-naltrexol is given orally, intravenously, intranasally, transdermally or subcutaneously.

5. A dosage form, comprising 6β-naltrexol, or a derivative or analog thereof in equipotent dosages, at a dosage of from 0.001 to 3.0 mg.

6. The dosage form according to claim 5, wherein said dosage form is an oral, intravenous, intranasal, transdermal or subcutaneous dosage form.

7. The dosage form according to claim 5, wherein said dosage form is an oral dosage form.

8. The dosage form according to claim 5, wherein said dosage of 6β-naltrexol or a derivative or analog thereof is from 0.001 to 0.25 mg.

9. The dosage form according to claim 5, wherein said dosage of 6β-naltrexol or a derivative or analog thereof is from 0.25 to 0.50 mg.

10. The dosage form according to claim 5, wherein said dosage of 6β-naltrexol or a derivative or analog thereof is from 0.50 to 0.75 mg.

11. The dosage form according to claim 5, wherein said dosage of 6β-naltrexol or a derivative or analog thereof is from 0.75 to 1.0 mg.

12. The dosage form according to claim 5, wherein said dosage of 6β-naltrexol or a derivative or analog thereof is from 1.0 to 1.25 mg.

13. The dosage form according to claim 5, wherein said dosage of 6β-naltrexol or a derivative or analog thereof is from 1.25 to 1.5 mg.

14. The dosage form according to claim 5, wherein said dosage of 6β-naltrexol or a derivative or analog thereof is from 1.5 to 1.75 mg.

15. The dosage form according to claim 5, wherein said dosage of 6β-naltrexol or a derivative or analog thereof is from 1.75 to 2.0 mg.

16. The dosage form according to claim 5, wherein said dosage of 6β-naltrexol or a derivative or analog thereof is from 2.0 to 3.0 mg.

17. The method according to claim 1, wherein said derivative or analog is 6β-naltrexamide, 6α-naltrexol, 6α-naloxol, 6β-naloxol, 6α-naltrexamine, 6β-naltrexamine, 6α-naloxamine, or 6β-naloxamine, or pegylated derivatives thereof.

18. The dosage form according to claim 5, wherein said derivative or analog is 6β-naltrexamide, 6α-naltrexol, 6α-naloxol, 6β-naloxol, 6α-naltrexamine, 6β-naltrexamine, 6α-naloxamine, or 6β-naloxamine, or pegylated derivatives thereof.

19. A pharmaceutical composition, comprising 6β-naltrexol, or a derivative or analog thereof at an equipotent dosage, at a dosage of from 0.001 to 3.0 mg, and an opioid analgesic at a dosage sufficient to treat pain.

20. The pharmaceutical composition according to claim 19, wherein said derivative or analog is 6β-naltrexamide, 6α-naltrexol, 6α-naloxol, 6β-naloxol, 6α-naltrexamine, 6β-naltrexamine, 6α-naloxamine, or 6β-naloxamine, or C-6 derivatives thereof, such as carboxyl esters, amides, or ethers (including pegylated derivatives), or reduced naltrexol or naloxol with a —C-6H₂ substitution.

21. A method of treating pain, comprising the step of administering 6β-naltrexol, or a derivative or analog thereof, in an amount of from 0.001 to 3.0 mg, and a therapeutically effective amount of an opioid analgesic to an individual in need thereof.

22. The method according to claim 21, wherein said derivative or analog is 6β-naltrexamide, 6α-naltrexol, 6α-naloxol, 6β-naloxol, 6α-naltrexamine, 6β-naltrexamine, 6α-naloxamine, or 6β-naloxamine, or C-6 derivatives thereof, such as carboxyl esters, amides, or ethers (including pegylated derivatives), or reduced naltrexol or naloxol with a —C-6H₂ substitution.

23. A method of identifying long-acting, neutral μ opioid antagonists binding with high affinity to MOR-μ* without blocking MOR-μ* signaling, acting at low doses selectively as modulators of dysregulated opioid signaling, comprising the step of gradually reducing the level MOR-μ* by accelerating reversal of MOR-μ* to the resting or reserve MOR states.
